(19) United States
(12) Patent Application Publication
(10) Pub. No.: US 2016/0207925 A1
(43) Pub. Date: Jul. 21, 2016

(54) L-METHYLFOLATE SALT PREPARATIONS, MEDICAMENTS, AND NUTRITIONAL SUPPLEMENTS COMPRISING SUCH SALTS THEREOF

(71) Applicant: Gianni Fracchia, Mosman (AU)

(72) Inventor: Gianni Fracchia, Mosman (AU)

(21) Appl. No.: 14/144,619

(22) Filed: Dec. 31, 2013

(51) Int. Cl. C07D 475/04; C07C 231/12; C07C 277/00; C07C 279/14; C07C 237/22

(52) U.S. Cl. CPC C07D 475/04; C07C 279/14; C07C 237/22; C07C 277/00; C07C 231/12; A23L 1/296; C07B 2200/07; A23V 2002/00

(57) ABSTRACT

The present invention provides salts of L-methylfolate of formula I,

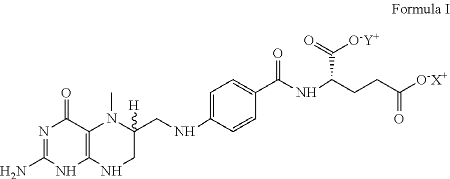

wherein X and Y both are elected from H, amino acids, and amine compounds, wherein both X and Y are not H in a single compound, i.e. when X═H, Y is not H and when Y═H, X is not H.

The invention also relates to a process for the preparation of salts of l-methylfolate. Furthermore, the invention also relates to medicaments and nutritional supplements comprising salts of l-methylfolate.

### FIELD OF INVENTION

The present invention is directed to salts of L-methylfolate. The invention also relates to a process for the preparation of salts of l-methylfolate. Furthermore, the invention also relates to medicaments and nutritional supplements comprising salts of l-methylfolate.

### BACKGROUND OF INVENTION

Folic acid, N-[4-[[(2-amino-1,4-dihydro-4-oxo-6-pteridinyl)methyl]amino]benzoyl]-L-glutamic acid is a water-soluble synthetic form of Vitamin B9 and the precursor of dihydrofolic acid and tetrahydrofolic acid. It is essential that folic acid is reduced to its metabolically active tetrahydrofolate form within the cell. Regular folic acid does not have coenzyme activity and undergoes a four-step enzymatic conversion process to achieve l-methylfolate—the active form of folic acid used by the body. The four-step enzymatic conversion process consists of: folic Acid converting into dihydrofolate (DHF), DHF converting into Tetrahydrofolate (THF), THF converting into 5,10-methylene THF, and finally, 5,10-methylene THF converting into l-methylfolate.

Methylenetetrahydrofolate reductase (MTHFR) is the enzyme responsible for catalyzing the conversion of 5,10-methylenetetrahydrofolate to 5-methyltetrahydrofolate, a cosubstrate for the conversion of the amino acid homocysteine to the amino acid methionine. Methionine is used by the human body to build proteins, process lipids and fats, reduce inflammation, utilize antioxidants, etc. Reduced MTHFR enzyme, a defective MTHFR enzyme, and gene variations, may result in increased: homocysteine levels, risk of neural tube defects, risk of birth defects, risk of coronary heart disease, risk of dementia, etc.

A MTHFR genotype variation in a person results in an inefficiency to convert regular folic acid to l-methylfolate. Thus, there is a need to develop medicaments and nutritional supplements comprising l-methylfolate which can be immediately absorbed by the body and are up to 700% more bioavailable than regular folic acid. It is known in the art that the S form of 5-methyltetrahydrofolic acid is the only active form; the R form is biochemically inactive and is excreted via the kidneys.

Folic acid offers various advantages and benefits for the human body. Folic acid plays a critical role in preventing, managing, or treating: depression, neural tube defects, pregnancy, homocysteine levels, cardiovascular concerns, etc. It is therefore evident that l-methylfolate offers similar advantages, with greater absorption and bioavailability.

U.S. Pat. No. 6,441,168 discloses 5-methyl-(6R,S)—, 5-methyl-(6S)—, or 5-methyl-(6R)— tetrahydrofolic acid said crystalline salt comprising a water of crystallization of at least one equivalent per equivalent of 5-methyl tetrahydrofolic acid.

U.S. Pat. No. 7,947,662 discloses a folic acid salt comprising: a moiety selected from D-glucosamine or D-galactosamine, and a folate or a reduced folate moiety selected from the group consisting of -folate, -dihydrofolate, and -tetrahydrofolate, wherein the folate or reduced folate moiety is unsubstituted or substituted with a moiety selected from the group consisting of 5-methyl-, 5-formyl-, 10-formyl-, 5,10-methylene-, and 5,10-methenyl, the compound, whenever contemplated, being in a (6R,S), (6S), or a (6R) configuration, or a salt thereof.

Neither U.S. Pat. No. 6,441,168 nor U.S. Pat. No. 7,947,662 discloses a compound which discloses or teaches near to a folate compound forming a salt with an amino acid or a nitro group. Thus, there arises a need to develop folate salts with higher absorption and high bio-availability.

### SUMMARY OF THE INVENTION

An objective of the present invention is to provide salts of L-methylfolate of formula I.

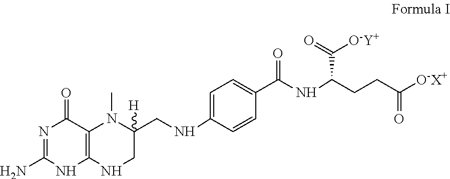

Another objective of the present invention is to provide a process for the preparation of salts of L-methylfolate of formula I.

Yet another objective of the present invention is to provide a simple, inexpensive, and efficient process, for the preparation of salts of L-methylfolate of formula I.

Yet another objective of the present invention is to provide salts of L-methylfolate of formula I which have high bioavailability.

Yet another objective of the present invention is to provide salts of L-methylfolate which have higher stability and shelf life.

Yet another objective of the present invention is to provide a solvent-free process for the preparation of salts of L-methylfolate of formula I.

Yet another objective of the present invention is to provide a medicament/nutritional supplement comprising at least one of the compounds of formula I.

### DETAILED DESCRIPTION OF THE PREFERRED EMBODIMENTS

The present invention provides salts of L-methylfolate of formula I,

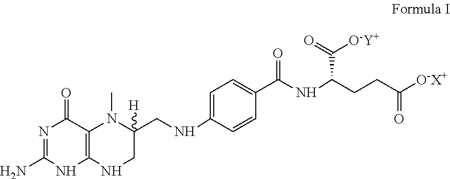

wherein X and Y both are elected from H, amino acids, and amine compounds, wherein both X and Y are not H in a single compound, i.e. when X═H, Y is not H and when Y═H, X is not H.

L-methylfolate has a negative charge. The bonding of l-methylfolate to a positive counter ion of is required to deliver chemical and water solubility, and increased absorption and bioavailability. According to the instant invention, the counter positive ion is provided by amino acids and amine compounds. X and Y represent the positive counter ion of preferably amino acids and amine compounds.

According to the invention, X and Y are selected from a group consisting of H, amino acids and amines.

The amino acids are selected from a group consisting of: glycine, alanine, valine, leucine, isoleucine, methionine, phenylalanine, tryptophan, proline, serine, threonine, cysteine, tyrosine, aspraginine, glutamine, aspartate, glutamate, lysine, arginine and histidine.

The amines are selected from a group consisting of: diethylamine, ammonium, tromethamine, primary, secondary and tertiary amines and basic amino carboxylic acids, especially selected from the group consisting of cyclohexyl amine, diisopropyl amine, benzyl amine, ammonia, ethanol amine, triethanol amine, 2-dimethyl amino-ethanol, tert.-butylamine, and the kind.

In an embodiment of the invention, preferred amino acids are arginine and asparagine.

Looking now more specifically to the preferred embodiments, one aspect of the invention is salts of l-methylfolate of structure I. In the above formula X and Y are H, an amino acid, or an amine compound, provided that when X is H, Y is not H and that when Y is H, X is not H. Accordingly, one of the O^{—} ion of the compound of l-methylfolate of formula I should necessarily be substituted by a counter ion of an amino compound selected from X and Y.

In another embodiment of the invention, the compound of formula I is a salt of substituted l-methylfolate, wherein the substituents are alkyl groups selected from alkyl chain compounds having 1 to 4 carbon atoms. It is to be noted that the substituents should not affect or reduce the advantageous property of the salts of l-methylfolate of formula I. The substituents should not compromise the advantages of salts of l-methylfolate.

In the first inventive process for the preparation of the salts of l-methylfolate, an aqueous solution of an amino salt is added to methyl-l-tetrahydrofolic acid under a nitrogen atmosphere. The obtained solution is dehydrated through lyophilization, producing a salt of l-methylfolate of formula I

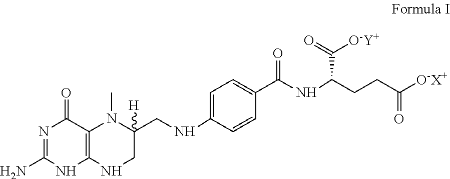

In an embodiment of the invention, the aqueous solution of an amino salt has a concentration have a concentration of salt to water ranging from 5.9/55 gm/ml to 5.9/85 gm/ml.

In the second inventive process for the preparation of the salts of l-methylfolate, an aqueous solution of an amino salt is added to methyl-I-tetrahydrofolic acid under a nitrogen atmosphere. The obtained solution is dehydrated through spray drying to obtain a salt of l-methylfolate of formula I.

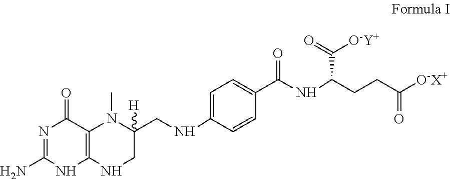

In the third inventive process for the preparation of the salts of l-methylfolate, an aqueous solution of an ammonium salt is added to methyl-l-tetrahydrofolic acid under a nitrogen atmosphere. The obtained solution is evaporated under reduced pressure to obtain a salt of l-methylfolate of formula I.

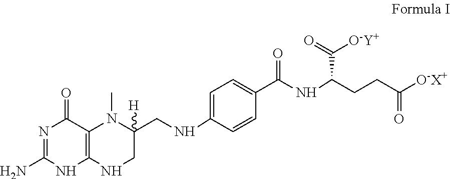

After obtaining the l-methylfolate compounds by the process steps mentioned above, the compounds are tested by High-Performance Liquid Chromatography (HPLC)to confirm the purity.

In an embodiment of the invention, the ratio of amount of amino salt to l-methylfolate is 1:10 to 5:1 w/w.

In another aspect of the invention there is provided a medicament comprising salts of l-methylfolate of formula-I for use as a supplement in: preventing, managing, or treating: prenatal periods, neural tube defects, homocysteine levels, homocysteine conditions, depression, dementia, and cardiovascular concerns. The medicament is characterized in that it contains at least one active compound of at least one of the possible compounds of formula I.

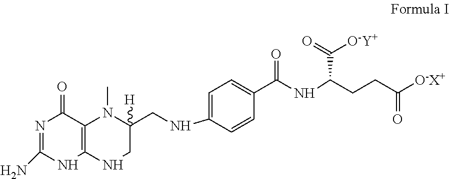

An amount of salt of l-methylfolate is preferably present in an amount from 100 mcg to 7500 mcg, preferably from 600 mcg to 1000 mcg, per dose unit, and the medicament is preferably in the form of a parenteral and/or oral pharmaceutical preparation.

Another aspect of the invention relates to the use of the salts of l-methylfolate of formula I, in a single form or in any combination for the preparation of a medicament for the management or treatment of a disease and/or use as a nutritional supplement.

According to an embodiment of the invention, the nutritional supplement is preferably in the form of a parenteral and/or oral pharmaceutical preparation.

The following examples illustrate the present invention.

All ingredients used for performing experiments have greater than 99% purity.

### EXAMPLE 1

Preparation of (6S)-5-Methyltetrahydrofolate L-Arginine Salt

The portion-wise addition of 70 ml of an aqueous solution of 1-arginine (5.9 g) to 7.75 g of methyl-l-tetrahydrofolic acid was stirred at room temperature and completely dissolved under a nitrogen atmosphere. The obtained solution was dehydrated through lyophilization, producing 14.5 g of creamy to light brown colored (6S)-5-methyltetrahydrofolate 1-arginine salt. HPLC analysis on the dry product calculated 57.15% 5-methyltetrahydrofolic acid.

### EXAMPLE 2

Preparation of (6S)-5-Methyltetrahydrofolate L-Arginine Salt

The portion-wise addition of 70 ml of an aqueous solution of 1-arginine (5.9 g) to 7.75 g of methyl-l-tetrahydrofolic acid was stirred at room temperature and completely dissolved under a nitrogen atmosphere. The obtained solution was dehydrated through spray-drying, producing 14.8 g of creamy to light brown colored (6S)-5-Methyltetrahydrofolate L-Arginine Salt. HPLC analysis on the dry product calculated 56.71% 5-methyltetrahydrofolic acid.

### EXAMPLE 3

Preparation of (6S)-5-Methyltetrahydrofolate L-Arginine Salt

The portion-wise addition of 70 ml of an aqueous solution of 1-arginine (5.9 g) to 7.75 g of methyl-l-tetrahydrofolic acid was stirred at room temperature and completely dissolved under a nitrogen atmosphere. The obtained solution was evaporated under reduced pressure at a temperature of 100° C./212° F., producing 14.2 g of creamy to light brown colored (6S)-5-Methyltetrahydrofolate L-Arginine Salt. HPLC analysis on the dry product calculated 55.27% 5-methyltetrahydrofolic acid.

### EXAMPLE 4

Preparation of (6S)-5-Methyltetrahydrofolate L-Asparagine Salt

The portion-wise addition of 70m1 of an aqueous solution of 1-asparagine (5.9 g) to 7.75 g of methyl-l-tetrahydrofolic acid was stirred at room temperature and completely dissolved under a nitrogen atmosphere. The obtained solution was dehydrated through lyophilization, producing 14.7 g of creamy to light brown colored (6S)-5-Methyltetrahydrofolate L-Asparagine Salt. HPLC analysis on the dry product calculated 55.88% 5-methyltetrahydrofolic acid.

### EXAMPLE 5

Preparation of (6S)-5-Methyltetrahydrofolate L-Asparagine Salt

The portion-wise addition of 70 ml of an aqueous solution of l-asparagine (5.9 g) to 7.75 g of methyl-l-tetrahydrofolic acid was stirred at room temperature and completely dissolved under a nitrogen atmosphere. The obtained solution was dehydrated through spray-drying, producing 14.9 g of creamy to light brown colored (6S)-5-Methyltetrahydrofolate L-Asparagine Salt. HPLC analysis on the dry product calculated 56.33% 5-methyltetrahydrofolic acid.

### EXAMPLE 6

Preparation of (6S)-5-Methyltetrahydrofolate L-Asparagine Salt

The portion-wise addition of 70 ml of an aqueous solution of l-asparagine (5.9 g) to 7.75 g of methyl-l-tetrahydrofolic Acid was stirred at room temperature and completely dissolved under a nitrogen atmosphere. The obtained solution was evaporated under reduced pressure at a temperature of 100° C./212° F., producing 14.4 g of creamy to light brown colored (6S)-5-Methyltetrahydrofolate L-Asparagine Salt. HPLC analysis on the dry product calculated 55.90% 5-methyltetrahydrofolic acid.

### METHOD SUMMARY

| | (6S)-5- | (6S)-5- | (6S)-5- | (6S)-5- | (6S)-5- | (6S)-5- |
|---|---|---|---|---|---|---|
| | MTHF L- | MTHF L- | MTHF L- | MTHF L- | MTHF L- | MTHF L- |
| | Arginine | Arginine | Arginine | Asparagine | Asparagine | Asparagine |
| | Salt | Salt | Salt | Salt | Salt | Salt |
| Method | Lyophilized | Spray- | Reduced | Lyophilized | Spray- | Reduced |
| | | Dried | Pressure | | Dried | Pressure |
| 5-MTHF | 57.15% | 56.71% | 55.27% | 55.88% | 56.33% | 55.09% |
| (HPLC) | | | | | | |

While there are shown and described present preferred embodiments of the invention, it is to be distinctly understood that the invention is not limited thereto, but may be otherwise variously embodied and practiced within the scope of the following claims.

What is claimed is:

1. A salt of l-methylfolate of formula I, 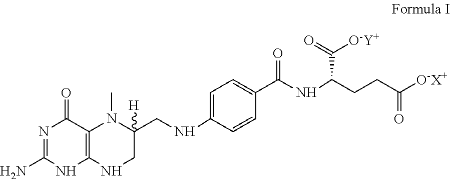
wherein X and Y both are selected from a group consisting of H, amino acids, and amine compounds, wherein when X═H, Y is not H and when Y═H, X is not H.

2. The salt of l-methylfolate according to claim 1, wherein the amino acids are selected from a group consisting of: glycine, alanine, valine, leucine, isoleucine, methionine, phenylalanine, tryptophan, proline, serine, threonine, cysteine, tyrosine, aspraginine, glutamine, aspartate, glutamate, lysine, arginine and histidine.
The salt of l-methylfolate according to claim 1, wherein the amines are selected from a group consisting of: diethylamine, ammonium, tromethamine, primary amines, secondary amines, and tertiary amines, and basic amino carboxylic acids, especially selected from the group consisting of, but not limited to: cyclohexyl amine, diisopropyl amine, benzyl amine, ammonia, ethanol amine, triethanol amine, 2-dimethyl amino-ethanol, tert.-butylamine, and the kind.

3. A process for preparation of salts of l-methylfolate of formula I 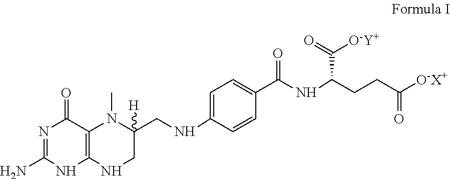
with a said process comprising adding an aqueous solution of an amino salt to methyl-l-tetrahydrofolic acid under a nitrogen atmosphere followed by dehydration to obtain a salt of l-methylfolate of formula I.

4. The process for preparing a salt of l-methylfolate according to claim 4, wherein dehydration is through lyophilization, spray drying, or evaporation under reduced pressure.

5. The process for preparing a salt of l-methylfolate according to claim 4, wherein the aqueous solution of an amino salt has a concentration of 5.9/55 gm/ml to 5.9/85 gm/ml.

6. The process for preparing a salt of l-methylfolate according to claim 4, wherein the ratio amount of an amino salt to l-methylfolate is between 1:10 to 5:1.

7. A medicament comprising a salt of l-methylfolate of formula I

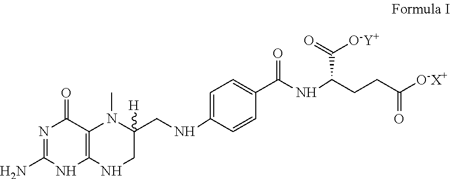

8. A nutritional supplement comprising a salt of l-methylfolate of formula I

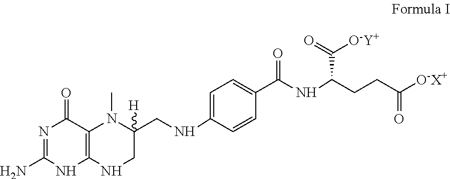

9. The medicament according to claim 9, wherein an amount of salt of l-methylfolate formula I ranges from 100 mcg to 7500 mcg, preferably from 600 mcg to 1000 mcg, per dose unit.

10. The medicament according to claim 9, wherein the medicament is preferably in the form of a parenteral and/or oral pharmaceutical preparation.

11. The nutritional supplement according to claim 9, wherein the nutritional supplement is preferably in the form of a parenteral and/or oral pharmaceutical preparation. [chemical structure]
